(19) United States
(12) Patent Application Publication
(10) Pub. No.: US 2026/0095010 A1
(43) Pub. Date: Apr. 2, 2026

(54) INSTALLATION TOOL SET FOR PRODUCING A HEAD PART, AND METHOD FOR PRODUCING A HEAD PART USING THE INSTALLATION TOOL SET

(71) Applicant: Neuroloop GmbH, Freiburg (DE)

(72) Inventors: Fabian Kimmig, Freiburg (DE); Tim Boretius, Freiburg (DE); Daniel Kempter, Freiburg (DE)

(21) Appl. No.: 19/114,865

(22) PCT Filed: Sep. 6, 2023
(86) PCT No.: PCT/EP2023/074428

§ 371 Date: Mar. 25, 2025

(30) Foreign Application Priority Data
Sep. 27, 2022 (DE) 10 2022 124 787.2

(51) Int. Cl. H01R 43/20; A61N 1/375; H01R 24/58; H01R 43/00

(52) U.S. Cl. CPC H01R 43/20; A61N 1/3752; H01R 24/58; H01R 43/005; H01R 2201/12

(57) ABSTRACT

An installation tool set for producing an implantable medical device includes an installation tool with a rod longitudinal axis and an axially extending rod surface, along which a local recess is introduced. A fixing body defines a through channel, through which the installation tool is passable along a first rod section and into which an opening opens. The opening is oriented orthogonally to the through channel, around which an internal thread is arranged within the fixing body. An adjusting rod has a joining contour that is counter-contoured relative to the local recess in the installation tool and a counter-thread engageable with the internal thread of the fixing body. A receiving structure defines a recess into which the installation tool is inserted. The fixing body, an electrically conductive contact ring, and an electrically insulating, elastically deformable seal ring are arranged coaxially and in axially serial sequence along the installation tool.

### CROSS-REFERENCE TO RELATED APPLICATIONS

This application is the United States national stage entry of International Application No. PCT/EP2023/074428, filed on Sep. 6, 2023, and claims priority to German Application No. 10 2022 124 787.2, filed on Sep. 27, 2022. The contents of International Application No. PCT/EP2023/074428 and German Application No. 10 2022 124 787.2 are incorporated by reference herein in their entireties.

### FIELD

Described are an installation tool set for producing a head part of an implantable medical device, as well as a method for producing a head part using the installation tool set.

### BACKGROUND

Implantable medical devices for the purpose of electrical stimulation of local intracorporeal areas, implantable pulse generators (IPG) for short, for example for cardiac defibrillation, pacemaker or resynchronisation applications, for therapeutic neurostimulation measures, brain stimulation or vagus nerve stimulation to name but a few, generally comprise a closed housing in which the components for electrical pulse generation are contained, such as at least one energy source and an electrical circuit structure connected thereto. In addition, directly adjoining the housing is a so-called head part in which an electrical contact device connected to the energy source and/or the circuit structure is integrated and into which a plug device, closing off the head part in a fluid-tight manner, can be fitted and contacts electrical supply and discharge lines for the purpose of the intracorporeal local application of the electrical stimulation signals as well as, if applicable, the supply of locally picked-up electrical signals to the electrical circuit structure present in the housing.

Described in document EP 2 134 418 B1 is a head part of the type in question of an implantable medical device which comprises two head part housing halves that can be joined together along a joining seam, introduced into each of which in serial sequence and separated by intermediate walls, are semi-cylindrically shaped recesses, into which in serially alternating order in each case, electrically conductive contact ring elements as well as electrically insulating seal rings are inserted. The head part formed by the two joined together head part housing halves thus comprises an arrangement of coaxially orientated and electrically insulated contact ring elements, for the electrical contacting of which a lateral access is provided in the head part, through which an electrical plug device can be inserted in a fluid-tight manner into a hollow space surrounded by all the annular contact ring elements.

Document DE 10 2012 010 901 A1 discloses a method for positioning and holding electrical contacts and seals within a head part for electrically contacting a medical implantable device. On one side, a blind hole-type boring is introduced into the head part housing made of a biocompatible and electrically insulating material, into which in alternating sequence electrically conductive contact rings and annular sealing elements are placed and jointly form a hollow space, into which a pin-shape plug device can be inserted. Within the head part, each of the individual annular contact rings is connected via a connecting line to electrical components located within the housing of the medical implantable device.

For the purpose of as durable fluid-tight sealing as possible and also electrical insulation of the individual contact ring elements with regard to each other, the alternating sequence of electrical contact ring elements and seal rings have to be fixed with the greatest possible axial joining force within the head part. For this, laborious installation measures are required, which can usually only be carried out manually and with considerable dexterity.

In document DE 20 2013 012 073 U1, a plug hole module assembly is disclosed, for the installation of which a number of contact rings and sealing elements are arranged in alternating sequence along a pin-shaped installation tool. By means of a tensioning device, all the contact rings and sealing elements mounted along the installation tool are clamped against each other under the application of an axial joining force. Serving to preserve the joining force is a sleeve element axially firmly mounted on the installation tool with a grub screw. In this tensioned state, a curable casting material that in the solidified state takes up the joining force is poured into the device.

Described in DE 10 2017 222 364 A1 is a method for producing a head part of an implantable medical device using a rod-shaped installation tool, along which in axially serially alternating sequence, electrically conductive contact ring elements as well as electrically insulating elastically deformable seal rings are arranged. To establish a joining force axially acting between the ring arrangement along the installation tool, the installation tool comprises on the one hand a mechanical stop and on the other hand at the end of the installation tool opposite this, an external thread onto which a nut, or suchlike, can be screwed. Through turning the nut about the external thread on the installation tool, the joining force acting along the axial ring sequence can be adjusted in fine increments. The thus assembled installation tool is then cast with a curable casting compound to form the head part, wherein out of the head part that forms after curing, only a small extension of the installation tool protrudes. By turning the installation tool about its longitudinal axis, this detaches from the hardened head part, wherein the nut remains in the head part as a lost component and the joining force acting between the axial ring sequence is intercepted by the casting material and is retained.

Disclosed in document DE 10 2019 203 273 A1 is a method for producing a medical implant that comprises a head part which has at least one blind hole-type recess in the form of an electrical plug contact socket, along which at least one electrically conductive contact element is arranged, and also envisages a supply part that is firmly connected to the head part and also has at least one electrical component, preferably in the form of a microcontroller and/or an electrical energy source which is electrically connected to at least one electrically conducting contact element by way of at least one electrical conductor structure. Comparable production methods are disclosed in documents DE 10 2020 112 084 A1 and DE 10 2020 205 350 A1.

### SUMMARY

The object of the invention is to specify an installation tool set for producing a head part of an implantable medical device as well as a method for producing a head part using an installation tool set in such a way that the production of head parts of the type in question should be possible in large numbers with high quality, consistent reproducibility as well as low fault rates or few to no reject parts.

Features further developing the inventive idea in an advantageous manner can be inferred from the further description with reference to the examples of embodiment.

The invention is based on the idea to simplify critical process and procedural steps in the production of head parts of the type in question, which, in particular relate to measures or working steps to be taken in the end phase shortly before completing the implantable medical device and as such can, in the event of occurring faults, result in maximum economic damage, especially if a large number of time-consuming or costly production steps have already been carried out. One of these critical measures relates to the separation of the aforementioned installation tool from the solidified casting material, wherein the plug socket formed like a freely accessible blind hole is created, into which a cone-shaped plug part can be fitted for electrical and mechanical contacting.

On the one hand, the installation tool set according to the invention represents simplified handling and installation of at least two, preferably a plurality of electrically conductive contact rings and electrically insulating, elastically deformable seal rings arranged serially in axially alternating sequence, and on the other hand makes possible the reliable and risk-free separation of individual installation tool components from the solidified casting material forming the completed head part.

The installation tool set according to the invention for producing a head part of an implantable medical device, which comprises a head part housing that can be made of a curable, pourable material and has a blind hole-type recess, along which at least one electrically conductive contact ring element as well as an electrically insulating seal ring are joined against each other in a force-fitting manner under the effect of an axial joining force, comprises the following tool components: a rod-shaped installation tool, a fixing body, an adjusting means as well as a receiving structure.

The rod-shaped installation tool has a rod longitudinal axis, as well as an axially extending rod surface, along which a local recess is introduced. A through channel passes through the fixing body with a channel longitudinal axis, through which the installation tool can be passed at least along a first rod section assigned thereto. In addition, the fixing body has one opening partially penetrating through this which opens into the through channel and had an opening longitudinal axis orientated orthogonally to the channel longitudinal axis of the through channel around which an internal thread is arranged within the fixing body.

The adjusting means has a joining contour ending at the end face that is freely accessible and is counter-contoured in relation to the local recess within the rod-shaped installation tool. In addition, the adjusting means comprises a counter thread that can be brought into engagement with the internal thread of the fixing body.

The receiving structure comprises a recess into which the rod-shaped installation tool, along which at least the fixing body as well as the at least one electrically conductive contact ring element as well as the at least one electrically insulating, elastically deformable seal ring are arranged coaxially and in axially serial sequence, can be inserted or are inserted under the effect of the axial joining force.

All the aforementioned tool components are separate components to be used and stocked, whose shape and size are matched to each other for installation purposes.

In a first installation step, at least the fixing body as well as at least one electrically conductive contact ring element and at least one electrically insulating, elastically deformable seal ring are arranged along the rod-shape installation tool. For this, the rod-shaped installation tool has a truncated cone-like rod end section, adjoining which is a first rod section with a constant first rod cross-section, which otherwise has an unstructured, smooth surface. In its longitudinal direction, the first rod section adjoins a second rod section, which in comparison with the first rod section has a larger rod cross-section and also, via an end face that extends orthogonally to the rod longitudinal axis, adjoins the first rod section and radially projects beyond the first rod section in an annular manner.

Preferably the arrangement of the fixing body as well as the at least one electrically conductive contact ring element and the at least one electrically insulating, elastically deformable seal ring along the rod-shaped installation tool takes place in such a way that initially an annular, elastically deformable seal body is pushed over the first rod end section of the installation tool and brought into contact on the end face of the second rod section. The fixing body is then arranged along the first rod section, axially directly adjoining the annular seal body. Preferably a plurality of contact ring elements and seal rings is then arranged along the installation tool, respectively in an axially alternating manner, forming a type of ring stack, which on one side adjoins the fixing body directly or indirectly via an additional elastic intermediate ring body.

In a next installation step, the adjusting means, preferably designed in a rod-like manner in the same way as the installation tool with its joining contour ending on the face side, is inserted into the opening in the fixing body that is arranged directly radially above the local recess in the axially extending rod surface of the first rod section of the installation tool. The local recess is preferably designed in the manner of a truncated cone-like groove encircling the first rod section in the circumferential direction, into which the joining contour of the adjusting means ending on the face side, engages locally and in a self-centring manner. The fitting process of the rod-like adjusting means into the opening of the fixing body takes place through rotation of the adjusting means about its rod longitudinal axis, through which the counter-thread applied on the adjusting means comes into contact with the internal thread provided in the area of the opening of the fixing body. In doing so, the joining contour of the adjusting means ending on the face side, enters into the local, groove-shaped, recess along the rod-shaped installation tool, forming a force and form-fitting connection. In this joining situation, the fixing body is axially firmly fixed or locked along the installation tool and is also in a non-rotatable manner about the rod longitudinal axis of the installation tool.

The local, preferably groove-shaped recess in the area of the first rod section of the installation tool is arranged at a defined first distance from the end face of the second rod section, so that on arranging the fixing body along the rod-shaped installation tool and fitting of the adjusting means into the opening of the fixing body, as described above, a second defined distance forms between the fixing body and the end face of the second rod section. Precisely this second distance maximally corresponds to an axial width measurement of the seal body that is designed in the manner of an elastomer seal ring and arranged around the rod-shaped installation tool axially adjoining on one side the fixing body and one the other side the end face of the installation tool. The seal body designed in the manner of an elastomer seal ring has a through opening whose shape and size are matched to the rod cross-section of the first rod cross-section of the installation tool. Through the axial surface contact on both sides of the ring-shaped seal body on the one hand with the end face of the installation tool, and on the other hand with the side of the fixing body, a fluid-tight joint is provided vis-à-vis a polymer, flowable casting material, i.e. when a flowable polymer casting material is poured around the assembly, penetration of casting material between the fixing body and the seal body as well as between the latter and the end face is ruled out.

In a further step, the installation tool together with at least the fixing body and at least one electrically conductive contact ring element and at least one electrically insulating, elastically deformable seal ring are inserted into the recess of the receiving structure, generating an axial joining force along at least one electrically conductive contact ring element and at least one electrically insulating, elastically deformable seal ring. After insertion into the recess of the receiving structure, the initially preferably manually generated joining force acting axially between the individual contact ring elements and seal rings arranged along the installation tool is taken up or absorbed by the receiving structure itself. For this purpose, the recess of the receiving structure, which is preferably made of a rigid or solidified material, comprises at least two axially opposite stop surfaces, on which on the one hand the rod end of the rod-shaped installation tool or an installation body optionally arranged thereon, and the fixing body on the other hand are supported directly or indirectly in such a way that at least one electrically conductive contact ring element and at least one electrically insulating, elastically deformable seal ring are subject to an axial joining force.

In a further step, a flowable and curable casting material is poured at least in parts around at least the receiving structure with the inserted installation tool and at least the fixing body as well as the at least one electrically conductive contact ring element as well as the at least one electrically insulating, electrically deformable seal ring as well as the adjusting means fitted by means of the joining body into the local recess of the rod-shaped installation means, in such a way that the installation tool and the adjusting means protrude from the casting material in some areas.

After hardening of the curable, preferably polymer material, the head part housing is formed, from which, in order to finish it, the rod-shaped installation tool and the rod-shaped adjusting means only have to be separated. Separation of the rod-shaped installation tool takes place using just axial tensile force along the installation tool, especially as the latter is only loosely or largely loosely fitted in the rest of the component assembly of the head part housing that is to be produced or has already been produced. To separate the adjusting means, the thread engagement between the adjusting means and fixing body needs to be loosened, followed by axial removal of the adjusting means out of the head part housing.

In a preferred embodiment, like the provision of a sealing body designed in the manner of an elastomer seal ring between the fixing body and the end face of the rod-shaped installation tool, a corresponding sealing body is also arranged between the fixing body and an end face assigned to the rod-shaped adjusting means. The sealing body serves to prevent the penetration of flowable casting material through the opening into the interior of the fixing body, and on the other hand, the sealing body makes it easier to separate the rod-shaped adjusting means from the solidified head part housing. For this, at its rod end, the adjusting means has the joining contour ending at the face side, to which adjoins a first rod-section of the adjusting means, along which the counter-thread in the form of an external thread is arranged. Directly bordering the first rod section of the adjusting means is a second rod section, which has a larger rod cross-section compared to the first rod section and an annular end face that faces the first rod section and radially surrounds it.

The fixing body is preferably cube or cuboid in shape and has at least two opposite lateral surfaces, a first and a second lateral surface, through which the through channel passes and each has a flat surface area orientated in parallel to each other. The fixing body also has a third lateral surface with a flat surface area which is orthogonally orientated with regard to the first and second lateral surfaces and through which the opening passes. In the case of complete fitting of the rod-shaped adjusting means within the opening of the fixing body, in which the joining contour on the face side engages in the local recess of the rod-shaped installation in a manner in which the contours match, a third distance remains between the end face of the adjusting means and the surface area of the third lateral surface of the fixing body, which maximally corresponds to an axial width dimension of an elastically deformable sealing body, which is arranged about the rod-shaped adjusting means, axially adjacent to the fixing body on the one hand and the end face of the adjusting means on the other hand. By screwing the adjusting means into the joining body, the deformable fixing body, which is preferably slightly over-dimensioned relative to the third distance, is compressed.

Preferably, the casting process that forms the head part housing is carried out with a preferably pourable, polymer casting material in such a way that it is poured completely around the installation tool placed in the receiving structure, together with the components described above, without the casting material wetting the parts of both the installation tool and the adjusting means protruding from the seal bodies. In this way, the installation tool as well as the adjusting means can be separated without problems after hardening and production of the head part housing.

Through the contour-matching fitting of the joining contour of the adjusting means ending at the face side within the local recess along the rod surface of the rod-shaped installation tool, the axial position of the fixing body along the installation tool is determined, through which the seal body arranged on the installation tool experiences a defined specified axial compression pressure between the fixing body on the one side and the end face of the assembly tool on the other. Furthermore, in this way the joining force acting between the plurality of contact ring elements and seal rings arranged along the assembly tool in axially alternating sequence can be adjusted in a predefined and reproducible manner, which is maintained by the geometry of the recess within the receiving structure and, in particular, the axial distance between the two axially opposite stop surfaces

### BRIEF DESCRIPTION OF THE DRAWINGS

The invention is described below as an example, without restriction of the general inventive idea, by way of examples of embodiment with reference to the drawings, of which:

FIG. 1 shows the rod-shaped installation tool with a fixing block arranged thereon;

FIG. 2 shows a perspective view of a fixing block on an installation tool;

FIG. 3 shows a joint assembly of installation tool, fixing block and adjusting means;

FIGS. 4 and 5 show an illustration of the joint between the adjusting means and groove-shaped recess along the installation tool;

FIG. 6 shows a receiving structure with components fitted therein; and

FIG. 7 shows a completed head part housing on an implantable medical device.

### DETAILED DESCRIPTION

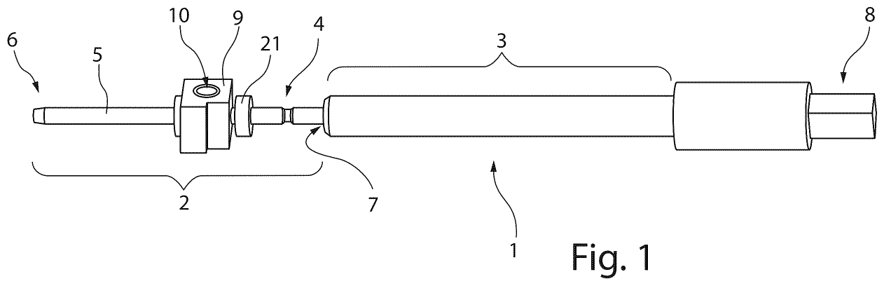

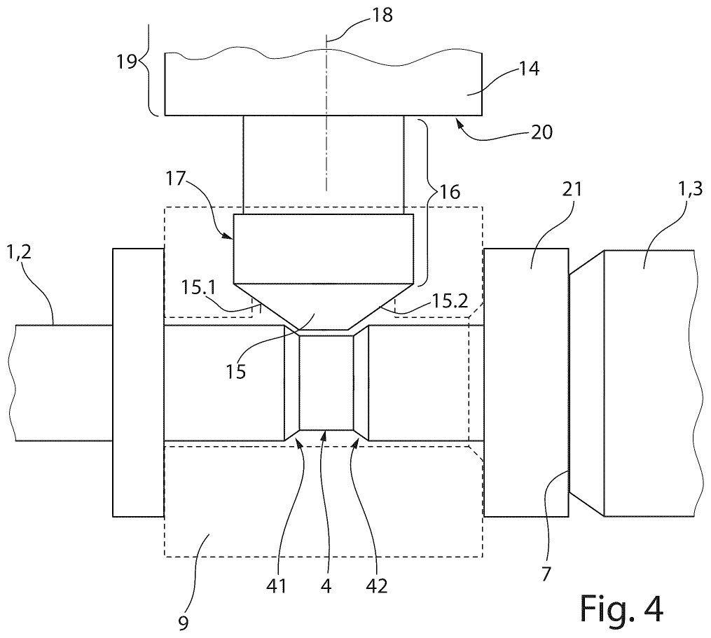

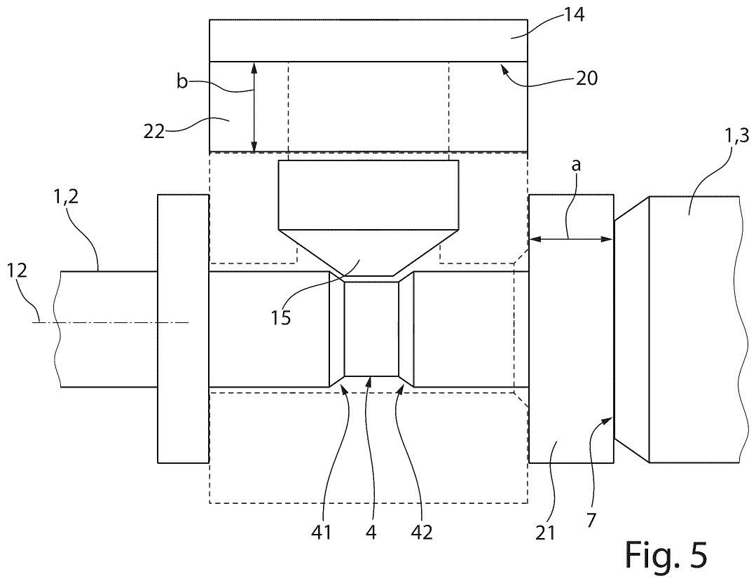

FIG. 1 shows a rod-shaped installation tool 1 with a first rod section 2 and adjoining it a second rod section 3. Both rod sections 2, 3 have a circular rod cross-section, wherein the first rod cross-section along the first rod section 2 is smaller in dimensions than the second rod cross-section in the second rod section 3. Along the first rod section 2, there is a local recess 4 in the form of a circumferential, groove-like recess, which can also be seen in more detail in FIGS. 4 and 5. The groove-shaped recess 4 is designed in the manner of a swallowtail contour and has two lateral flanks 41, 42 that along the first rod section 2 are obliquely angled with regard to the otherwise smoothly formed rod surface 5, and further serve for the purpose of a centring and axial locking function.

The first rod section 2 is delimited on the one site by a conical blunt rod end 6 and on the other by an annular end face 7 radially projecting beyond the rod surface 5 of the first rod section 2.

The end opposite the conical blunt rod end 6 of the rod-shaped installation tool 1 has a mechanically stable interface 8 on which a tool contour is provided via which the tensile forces acting at least along the rod-shaped installation tool 1 can be introduced.

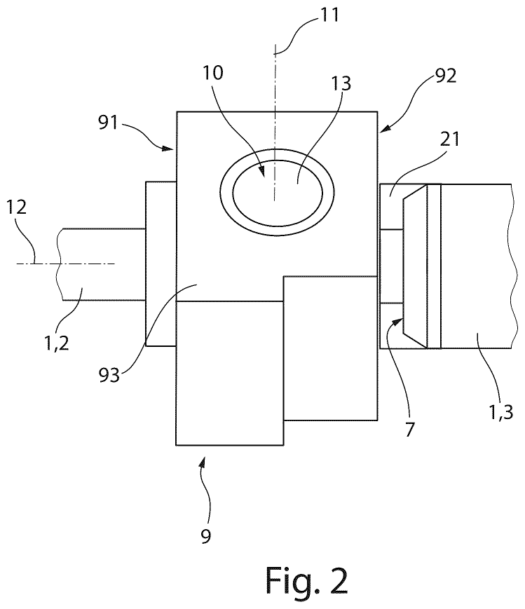

Furthermore, along the first rod section 2, a fixing body 9 is pushed on or arranged along the rod-shaped installation tool 1, which can also be seen in the perspective view in FIG. 2. The fixing body 9, which, like the rod-shaped installation tool 1, is made of a solid material, preferably of a metallic material, has a cube-shaped or cuboid basic shape which is completely penetrated by a through channel through which the rod-shaped installation tool 1 projects with its first rod section 2. The not further visible through channel passes through the two lateral surfaces 91, 92 of the fixing body 9 that are orientated in parallel to each other. In addition, the fixing body 9 has an opening 10, with an opening longitudinal axis 11 passing perpendicularly through the opening 10, which is orthogonal to the longitudinal axis of the through channel and thus to the longitudinal rod axis 12 of the installation tool 1. The opening 10 is adjacent to a flat lateral surface 93 of the fixing body 9, which is also orientated orthogonally to the lateral surfaces 91, 92.

Furthermore, an internal thread 13 is incorporated on the inside of the fixing body 9 around the opening longitudinal axis 11, directly adjacent to the opening 10.

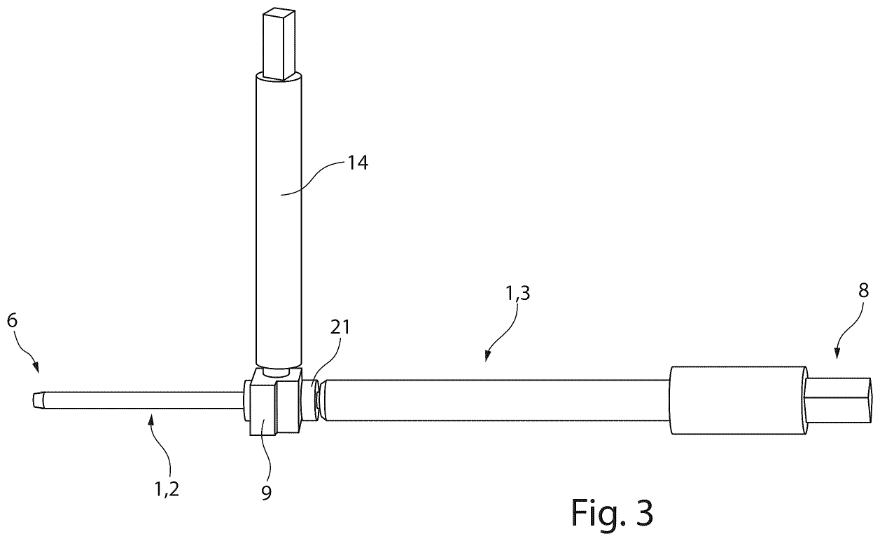

In FIG. 3, in addition to the rod-shaped installation tool 1 and the fixing body 9 arranged along the first rod section 2 of the installation tool 1, a rod-shaped adjusting means 14 is illustrated, which projects at the end into the opening 10 of the fixing body 9 and is in firm joining connection therewith.

FIGS. 4 and 5 are both detailed views for illustrating the joining connection between the adjusting means 14, the fixing body 9 as well as the installation tool 1.

At its rod end, the rod-shaped adjusting agent 14 has a face end joining contour 15, which is counter-contoured with regard to the groove-shaped, local recess 4. The face end joining contour 15 also has oblique side flanks 15.1, 15.2, whose slope corresponds to the side flanks 41, 42 of the local recess 4.

Directly adjoining the face end joining contour 15 is a first rod section 16 of the adjusting means 14, along which an external thread 17 is arranged that is designed as a counter-thread to the internal thread 13 within the fixing body 9 and can be brought into engagement therewith through rotation of the adjusting means 14 along its rod longitudinal axis 18.

Directly adjoining the first rod section 16 of the adjusting means 14 is a second rod section 19, which has a larger rod cross-section compared to the first rod section 16 so that an annular end face 20 facing the first rod section 16 and radially surrounding it is provided.

FIG. 5 illustrates the assembled state of the rod-shaped adjusting 14 means by way of the fixing body 9 on the rod-shaped installation tool 1. Here, the opening 10 of the fixing body 9 is radially centrically located above the local recess 4, into which the end face joining contour 15 is detachably securely joined in a form and force fitting manner by means of a threaded joint between the adjusting means 14 and the fixing body 9. The fixing body 9 is thus fixed axially firmly relative to the rod-shaped installation tool 1 on the one hand, and on the other hand fixed to the installation tool 1 in a non-rotational manner due to the contact forces prevailing between the face end joining contour 15 and the local recess 4.

The spatial arrangement of the recess 4 along the rod-shaped installation tool 1 relative to its face end 7, as well as the geometric lateral dimensions of the fixing body 9, are matched to each other in such a way that in the joined state, a distance a is provided between the lateral surface 92 of the fixing body 9 and the end face 7 of the installation tool 1. Similarly, a corresponding distance b is provided between the lateral surface 93 of the fixing body 9 and the end face 20 of the adjusting means 14. The two distances a, b allow the additional fitting of one sealing body 21, 22 respectively, which are each designed as elastomer seal rings. Thus, on the one hand, the sealing body 21 is mounted along the rod-shaped installation tool 1 and borders on one side on the lateral surface 92 of the fixing body 9 and on the other hand on the end face 7 of the rod-shaped installation tool 1 in a fluid-tight manner. In a similar way, the second sealing body 22 is mounted along the first rod section 16 of the adjusting means 14 and borders on one side on the lateral surface 93 of the fixing body 9 and on the other hand on the end face 20 of the adjusting means 14 in a fluid-tight manner. The distances a, b as well as the width dimensions of the annular seal bodies 21, 22 are coordinated with each other in such a way that through elastomeric compression of both seal bodies 21, 22, a fluid-tight surface seal is provided, at least with regard to a flowable, polymer casting material.

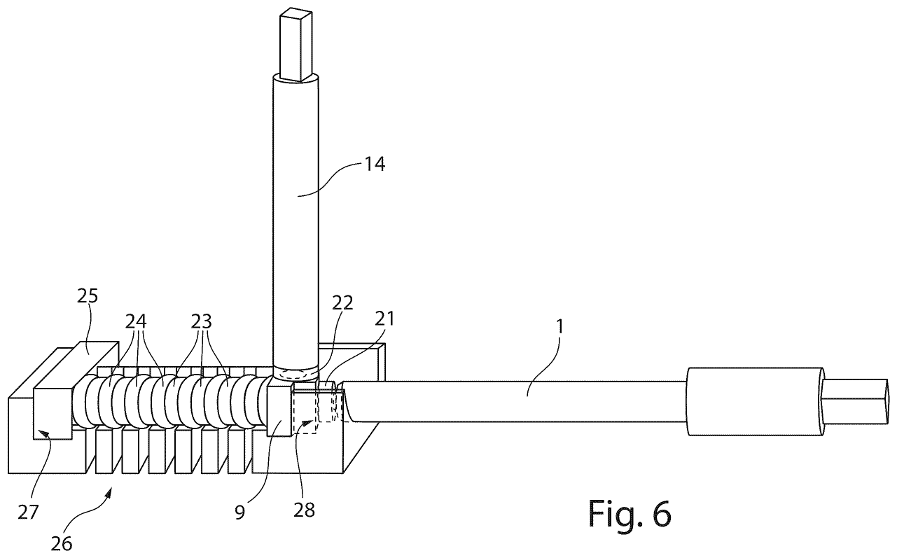

FIG. 6 shows an arrangement showing the assembled state of the adjusting means 14 by means of the fixing body 9 with the rod-shaped installation tool 1, wherein between the adjusting means 14 and the fixing body 9, the annular sealing body 22, and between the fixing body 9 and the rod-shaped installation tool 1, the annular sealing body 21 are respectively arranged under the effect of a pressing force.

In addition, along the first rod section 2 of the rod-shaped installation tool 1, a plurality of electrically conductive contact ring elements 23 as well as electrically insulating, elastically deformable seal rings 24 are arranged in axially alternating sequence. The conical blunt rod end 6 of the rod-shaped installation tool 1 is loosely inserted into an installation body 27 on one side, by means of which an axially acting joining force can be generated by manual axial compression of the serial axial sequence of contact ring elements and seal rings 23, 24. In this axially compressed state, the arrangement is inserted into the recess of a receiving structure 26, which essentially has two axially opposite stop surfaces 27, 28, on which on the one hand the installation body 25 and on the other hand the fixing body 9 can be brought into mutually supporting contact so that the receiving structure 26 is able to take up the joining force.

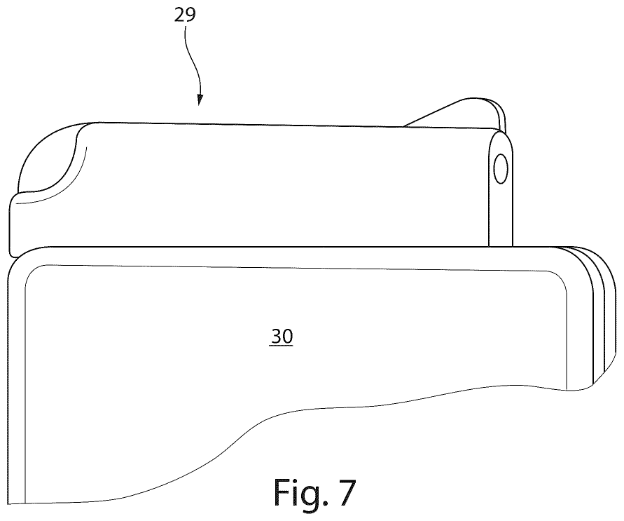

The recess otherwise incorporated within the receiving structure 26 is matched accordingly to the outer contour of the axial sequence of contact ring elements and seal rings 23, 24 as well as to the fixing body 9 in a counter-contoured manner. The receiving structure 26 preferably consists of a solidified casting material that is used to form the head part housing 29 shown in FIG. 7 by way of a pouring process. The pouring process surrounds the receiving structure 26 with all components fitted therein and preferably ends in a fluid-tight manner at the annular sealing bodies 21, 22, so that the second rod sections 3, 19 of the installation tool 1 and the adjusting means 14 are not surrounded by the casting material during the pouring process. This considerably facilitates the separation of the installation tool 1 and the adjusting means 14 from the assembly, so that as a result, the head part housing 29 that is shown in FIG. 7 mounted on a medically active implantable device 30 is obtained.

### LIST OF REFERENCE NUMBERS

1 Rod-shaped installation tool
2 First rod section
3 Second rod section
4 Local recess
41, 42 Oblique lateral flank
5 Rod surface
6 Rod end
7 End face
8 Interface
9 Fixing body
91,92,93 Lateral surface
10 Opening
11 Opening longitudinal axis
12 Through channel longitudinal axis, rod longitudinal axis
13 Internal thread
14 Adjusting means
15 End face joining contour
15.1, 15.2 Lateral flank
16 First rod section
17 External thread
18 Rod longitudinal axis
19 Second rod section
20 End face
21,22 Seal body
a, b Distance
23 Electrically conductive contact ring element
24 Electrically insulating, elastically deformable seal ring
25 Installation body
26 Receiving structure
27,28 Stop surface
29 Head part housing
30 Implantable device

1.-17. (canceled)

18. An installation tool set for producing a head part of an implantable medical device, the head part having a head part housing manufacturable from a curable, pourable material, the head part having a blind hole-type recess, along which in coaxial arrangement and axially serial sequence, at least one electrically conductive contact ring element and an electrically insulating seal ring are joined against each other in a force-fitting manner under an axial joining force, the installation tool set comprising:
a rod-shaped installation tool with a rod longitudinal axis and an axially extending rod surface, along which a local recess is introduced;
a fixing body, through which a through channel having a longitudinal channel axis passes, through which the rod-shaped installation tool is passable along at least a first rod section into which an opening at least partially penetrating the fixing body opens, the opening having an opening longitudinal axis orthogonally orientated to the longitudinal channel axis, around which an internal thread is arranged within the fixing body;
an adjusting rod with a joining contour ending at a face side that is counter-contoured with regard to the local recess in the rod-shaped installation tool, as well as a counter-thread which is engageable with the internal thread of the fixing body; and
a receiving structure defining a recess into which the rod-shaped installation tool is insertable under the axial joining force,
at least the fixing body, the at least one electrically conductive contact ring element, and the electrically insulating seal ring being arranged coaxially and in axially serial sequence along the rod-shaped installation tool.

19. The installation tool set according to claim 18, wherein:
the local recess is arranged in an area of the first rod section,
the first rod section having a first rod cross-section that is constant at all sections except for the local recess, the first rod section being delimited by a rod end section of the installation tool and by a second rod section with a second rod cross-section that is larger than the first rod cross-section, and
the second rod section adjoins the first rod section via an end face extending orthogonally to the rod longitudinal axis.

20. The installation tool set according to claim 19, wherein the rod end section of the installation tool is designed in a truncated cone-like manner and has an unstructured, smooth surface.

21. The installation tool set according to claim 19, wherein the local recess is arranged relative to the end face of the second rod section along the rod longitudinal axis in such a way that when the fixing body is arranged along the rod-shaped installation tool and the adjusting rod is fitted into the opening of the fixing body in such a way that the joining contour of the adjusting rod ending at the end face enters into the local recess along the rod-shaped installation tool and with this forms a force and form-fitting connection between the fixing body and between the fixing body and the end face of the second rod section of the installation tool a first distance is provided.

22. The installation tool set according to claim 21, wherein the first distance maximally corresponds to an axial width measurement of an elastically deformable seal body that is arranged around the rod-shaped installation tool axially adjoining on a first side the fixing body and on a second side the end face of the installation tool.

23. The installation tool set according to claim 22, wherein the elastically deformable seal body comprises a through opening having a shape and size that are matched to a rod cross-section of the first rod section of the installation tool.

24. The installation tool set according to claim 18, wherein the adjusting rod has a rod end that provides for the joining contour ending at a face end, a first rod section adjoining the joining contour, along which the counter-thread is arranged as an external thread, and also with a second rod section adjoining the first rod section, the second rod section having a larger rod cross-section compared to the first rod section and an end face facing the first rod section and radially surrounding the first rod section.

25. The installation tool set according to claim 18, wherein:
the fixing body is cube or cuboid in shape,
the fixing body comprises a first lateral surface and a second lateral surface opposite the first lateral surface,
the through channel passes through the first lateral surface and the second lateral surface,
the first lateral surface has a first flat surface area and the second lateral surface has a second flat surface area oriented parallel to the first flat surface area,
the fixing body further comprises a third lateral surface with a third flat surface area oriented orthogonally to the first lateral surface and the second lateral surface, the opening passing through the third lateral surface.

26. The installation tool set according to claim 25, wherein:
when the adjusting rod is completely fitted within the opening of the fixing body, in which an end face joining contour of the adjusting rod engages in the local recess of the rod-shaped installation tool in a manner in which contours match, an end surface of the adjusting rod has a second distance with regard to surface area of the third lateral surface of the fixing body.

27. The installation tool set according to claim 26, wherein the second distance maximally corresponds to an axial width measurement of an elastically deformable seal body that is arranged around the adjusting rod axially adjoining the fixing body on a first side and an end face of the adjusting rod on a second side.

28. The installation tool set according to claim 27, wherein the elastically deformable seal body comprises an elastomer seal ring with a through opening having a shape and size that are matched to a first rod cross-section of the adjusting rod.

29. The installation tool set according to claim 18, wherein the recess comprises at least two axially opposite stop surfaces, on which a rod end of the rod-shaped installation tool and the fixing body are supported directly or indirectly in such a way that the at least one electrically conductive contact ring element and the electrically insulating seal ring are subject to an axial joining force, that is taken up by the at least two axially opposite stop surfaces.

30. The installation tool set according to claim 18, wherein the local recess is designed as a truncated cone-like groove encircling the first rod section in a circumferential direction.

31. A method for producing a head part of an implantable medical device using the installation tool set according to claim 18, the head part having a head part housing manufacturable from a curable, pourable material, the head part having a blind hole-type recess, along which in coaxial arrangement and axially serial sequence, at least one electrically conductive contact ring element as well as an electrically insulating seal ring are joined against each other in a force-fitting manner under an axial joining force, the method comprising the steps of:
arranging at least the fixing body, the at least one electrically conductive contact ring element, and the electrically insulating seal ring along the rod-shaped installation tool;
fitting the joining contour of the adjusting rod ending at an end face into the local recess by engagement of the counter-thread of the adjusting rod with the internal thread of the fixing body;
inserting the rod-shaped installation tool together with at least the fixing body, the at least one electrically conductive contact ring element, and the electrically insulating seal ring into the recess of the receiving structure;
generating an axial joining force along the at least one electrically conductive contact ring element and the electrically insulating seal ring;
pouring a flowable and curable casting material at least in parts around at least the receiving structure with the installation tool and at least the fixing body, the at least one electrically conductive contact ring element, the electrically insulating seal ring, and the adjusting rod placed by the fixing body into the local recess of the rod-shaped installation tool, in such a way that the rod-shaped installation tool and the adjusting rod protrude from a casting material in parts;
hardening the curable, pourable material forming the head part housing; and
separating the rod-shaped installation tool and the adjusting rod from the head part housing.

32. The method according to claim 31, wherein, before arranging at least the fixing body, the electrically insulating seal ring is fitted along a first rod cross section of the rod-shaped installation tool and brought into contact on an end face of the rod-shaped installation tool.

33. The method according to claim 31, wherein:
after arranging at least the fixing body and before fitting the joining contour of the adjusting rod ending at an end face into the local recess in the rod-shaped installation tool, an installation body is mounted on a rod end section of the rod-shaped installation tool, which, together with the fixing body, axially delimits on both sides the at least one electrically conductive contact ring element as well as the electrically insulating seal ring along the rod-shaped installation tool, and
the installation body, the rod-shaped installation tool, the fixing body, the at least one electrically conductive contact ring element and the electrically insulating seal ring are inserted into the recess of the receiving structure.

34. The method according to claim 31, wherein:
before joining, the electrically insulating seal ring is fitted along the first rod section of the adjusting rod and brought into contact on an end face of the adjusting rod, and
fitting of the adjusting rod takes place in such a way that the electrically insulating seal ring applied to the adjusting rod for flowable casting material adjoins the fixing body and an end surface of the adjusting rod in fluid-tight manner.
